(19) United States
(12) Patent Application Publication
(10) Pub. No.: US 2007/0038037 A1
(43) Pub. Date: Feb. 15, 2007

(54) Method and apparatus for symptom-based order protocoling within the exam ordering process

(75) Inventors: Steven Fors, Chicago, IL (US); Khal A. Rai, Round Lake, IL (US); Mark Morita, Arlington Heights, IL (US)

Correspondence Address: MCANDREWS HELD & MALLOY, LTD, 500 WEST MADISON STREET, SUITE 3400, CHICAGO, IL, 60661, US

(73) Assignee: General Electric Company

(21) Appl. No.: 11/203,857

(22) Filed: Aug. 15, 2005

(51) Int. Cl. A61B 5/00; G06Q 10/00; G06F 19/00

(52) U.S. Cl. 600/300; 705/3; 705/2; 128/920

(57) ABSTRACT

A system and method to increase the efficiency of the healthcare system. Computer software for analyzing data and returning recommended procedure types based on the data. The data may include, for example, medical data, patient symptoms, patient vitals, patient history, chief complaint of a patient, intakes, outputs, physical information, medications, or demographic information and/or other information relevant to a patient's health. The data may be input from a user or information recommended from another source, such as a database. Computer software may analyze the data and return recommended procedure types based on the data. The analysis performed by the computer software may include mapping the data among potential procedures to determine which procedures should be performed based on the data. Procedures that are successfully mapped with the data may be recommended to a user and ordered from a user interface.

### BACKGROUND OF THE INVENTION

The present invention generally relates to computer software for medical systems. Particularly, the present invention relates to a system and method for computer software for analyzing data and returning recommended procedure types based on the data.

The current workflow of a medical examination of a patient is time consuming and inefficient for both the patient and the physician. Upon a patient's first visit with a physician, a patient generally provides background information about herself to the physician. For example, a patient may provide information such as family history, past problems, medications, or other information that captures the status of the patient's past and present health. A patient may then provide more specific information to a person, such as a nurse. The specific information may concern symptoms of an injury or illness. For example a nurse may gather information such as a chief complaint, vitals, intake, or outtake. A physician may then review the available information about a patient and decide whether further tests should be ordered.

The current process of ordering medical tests based on patient parameters such as symptoms, may be prone to human error. A physician generally either remembers the standard exams for given symptoms, or consults a medical reference to place orders for the most relevant exams. As the sophistication of medical technology increases, so does the number of available medical tests. It may be difficult for a physician to remember the available exams for a given set of symptoms, and thus a physician may not order a beneficial exam or order non-beneficial exams, both decreasing the efficiency of patient diagnosis and treatment. Additionally, a physician may not be aware of new tests or procedures available for a given set of symptoms and the physician may not order beneficial exams.

Accordingly, there is a need for a system and method to support a physician's decision to order examinations based on the data available. Such a system and method may minimize errors by a physician and increase the efficiency the healthcare system.

### SUMMARY OF THE INVENTION

Certain embodiments of the present invention provide a system for supporting a patient workflow. The system comprises a computer system for executing computer software. The computer software analyzes data and returns recommended procedure types based on the data. The data may include, for example, medical data, patient symptoms, patient vitals, or patient history. The data may also include, for example, one or more of the chief complaint of a patient, intakes, outputs, physical information, medications, or demographic information. The computer software may include a user interface. The user interface may have a user compartment, a patient information compartment, and an order entry compartment. The order entry compartment identifies the available procedure types. The order entry compartment may also provide for ordering the available procedure types.

Certain embodiments of the present invention provide a method for supporting a patient workflow using computer software. The method comprises receiving patient data, analyzing the patient data, and returning recommended procedure types based on the patient data. The patient data may include, for example, medical data, patient symptoms, patient vitals, or patient history. The patient data may also include, for example, one or more of the chief complaint of a patient, intakes, outputs, physical information, medications, or demographic information. The computer software may include a user interface. The user interface may have a user compartment, a patient information compartment, and an order entry compartment. The order entry compartment identifies the available procedure types. The order entry compartment may also provide for ordering the available procedure types.

Certain embodiments of the present invention may provide for a computer-readable storage medium including a set of instructions for a computer. The set of instructions comprises a receipt routine for receiving patient data, an analysis routine for analyzing the patient data, and a return routine for returning recommended procedure types based on the patient data. The patient data may include, for example, medical data, patient symptoms, patient vitals, or patient history. The patient data may also include, for example, one or more of the chief complaint of a patient, intakes, outputs, physical information, medications, or demographic information. The set of instructions may further include a user interface routine for operating a user interface. The user interface may have an order entry compartment.

### BRIEF DESCRIPTION OF THE DRAWINGS

FIG. 1 illustrates an example of a system for supporting a patient workflow in dance with an embodiment of the present invention.

FIG. 2 illustrates screen shot of a user interface that may be used in accordance an embodiment of the present invention.

FIG. 3 illustrates a method supporting a patient workflow using computer are in accordance with an embodiment of the present invention.

### DETAILED DESCRIPTION OF THE INVENTION

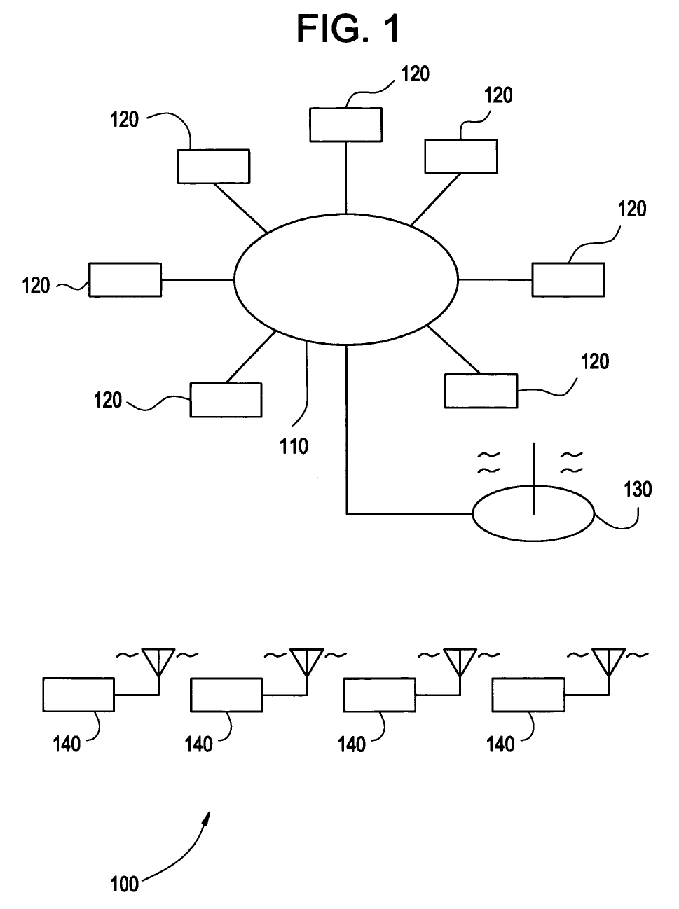

FIG. 1 illustrates a system 100 for supporting a patient workflow. The system 100 is example of a system that may be used in accordance with an embodiment of the present invention. As an example, the system 100 shows a central computer 110, a plurality of wired user computers 120, a plurality of wireless user computers 140 (collectively the “user computers 120, 140”), and a wireless base station 130. The system 100 is just an example and the system 100 may support wired and/or wireless communication with any number of user computers 120, 140 and any number of base stations 130. The system 100 may comprise various input devices, output devices, network connections, and connections to external equipment. Additionally, the components of the system 100 may be single units, separate units, may be integrated in various forms, and may be implemented in hardware and/or in software.

The central computer 110 may be a server type computer that may be composed of a single computer unit or a plurality of computer devices. The central computer 110 may be scalable according to the number of user computers 120, 140 the central computer 110 serves. In an embodiment, the central computer 110 may include, or be connected to, various processing components, data storage capabilities, and other computer devices. The central computer 110 may also be a part of an electronic network, such as an intranet or the Internet. In operation, the central computer 110 may store various information such as medical records for patients, communicate with user computers 120, 140, and process data, among other functions. The central computer 110 may be in electrical communication with a plurality of user computers 120, 140 through a wire and/or wirelessly.

The user computers 120 may be general personal computer types with, for example, input components of a mouse and keyboard. The user computers 120 may be in electrical communication with the central computer 110 through a wire, as shown in FIG. 1. The user computers 120 may be used to communicate with the central computer 110 and communicate over a network and/or the Internet with other computers and computer devices.

The wireless user computers 140 may be any device that can communicate wirelessly with the central computer 110. For example, the wireless user computers 140 may be laptop computers, tablet personal computers, personal digital assistants, or cell phones. The wireless user computers 140 may be in electrical communication with the central computer 110 through the wireless base station 130, as shown in FIG. 1. The wireless user computers 140 may be used to communicate with the central computer 110 and communicate over a network and/or the Internet with other computers and computer devices. The base station 130 may be any device which permits wireless communication of data.

An example of the system 100 may be a hospital computer system. The central computer 110 may be a central hospital computer and/or database. In an embodiment, the central computer 110 may store patient medical records, among other information, and may run various computer programs. The central computer 110 may be in communication with a plurality of user computers 120, 140 throughout the hospital and external networks, such as the Internet. The user computers 120 may communicate over wires throughout the hospital and the wireless user computers 140 may communicate wirelessly within range of a base station 130.

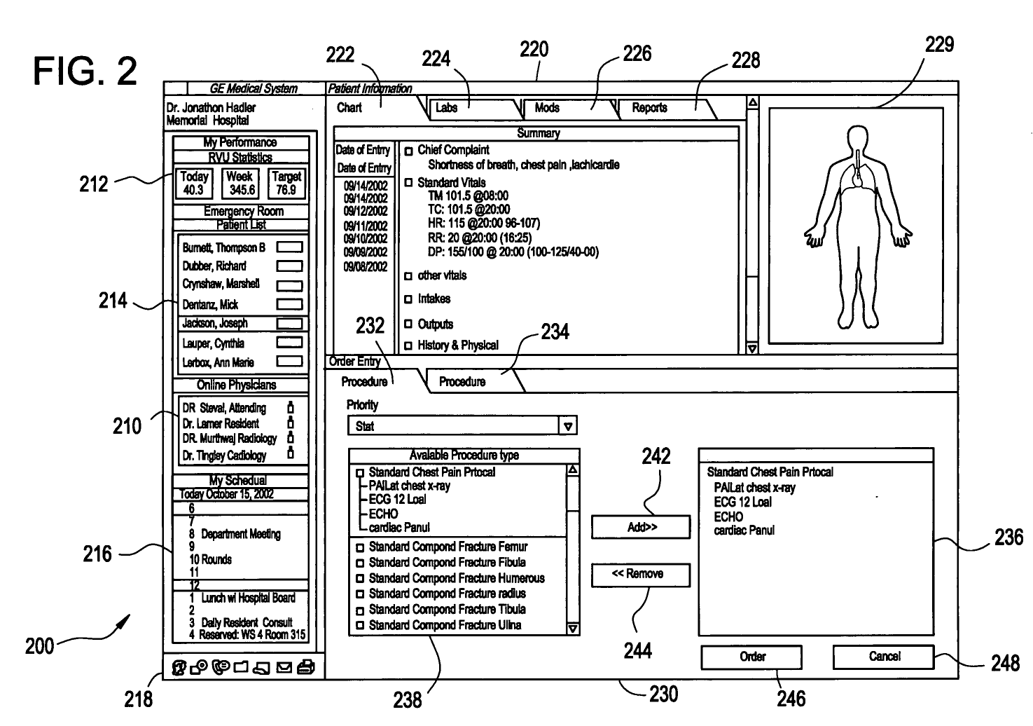

FIG. 2 illustrates a screen shot of decision support computer software 200 that may run on the system 100. The decision support computer software 200 may run on the central computer 110, the user computers 120, 140, or some combination thereof. As the computer software 200 runs on a networked system, the computer software may be periodically updated with current information, such as new examinations and/or procedures. The screen shot illustrated in FIG. 2 is generally an example of a user interface that may be displayed on the user computers 120, 140.

As shown, the screen shot of the decision support computer software 200 has three compartments. Compartment 210 organizes and displays information relevant to the individual user. For example, three sections of compartment 210 are shown. Sections 212, 214, and 216 provide information specific to the user. For example, section 214 provides a user with a list of patients and highlights the patient name for which the patient information 220 and order entry 230 compartments are active. Section 216 provides a user with a calendar function so the user may keep appointments and schedule in view. Section 218 provides links for a user to initiate other programs.

The patient information compartment 220 provides a user with data. The data may include, for example, medical data, patient symptoms, patient vitals, or patient history. The data may also include, for example, one or more of the chief complaint of a patient, intakes, outputs, physical information, medications, or demographic information. Other types of data may also be provided to the user and organized among the tabs. As shown, the patient information compartment 220 has four tabs: a chart tab 222, a lab tab 224, a meds tab 226, and a reports tab 228. Each tab may contain various information about the patient. For example, the reports tab 228 may contain reports on the patient. The meds tab 226 may contain medications prescribed, medications the patient is currently using, or medications the patient has used or been prescribed in the past, for example. The labs tab 224 may display information regarding current laboratory tests or previous laboratory tests, and may display the results of those tests, for example. The chart tab 222, shown in FIG. 2, may display information that would ordinarily be on a patient's chart. In an embodiment, the patient information compartment 220 may also display an image of the human body 229 with the area of suspected illness or injury highlighted.

Compartment 230 provides a user with order entry information. Compartment 230 has two tabs, procedure 232 and procedure 234. The procedure tabs 232 and 234 may be used for separate procedures for an individual patient. In an embodiment, any number of procedure tabs may be used. Compartment 230 also has two windows. Window 238 displays the available/recommended procedures to the user. In the embodiment shown in FIG. 2, the available/recommended procedures may be highlighted and expanded. A user may click the add button 242 to add the available/recommended procedures from the available procedure type window 238 to the requested procedure window 236. The window 236 then displays the requested procedures. The user may also remove a procedure from the requested procedure window 238 by highlighting the procedure and selecting the remove button 244. A user may order the procedures in the requested procedures window 236 by selecting the order button 246.

In operation of the system 100 executing computer software and user interface 200, a first user may input data about a patient into the system using the user computers 120, 140. The data may include, for example, medical data, patient symptoms, patient vitals, or patient history. The data may also include, for example, one or more of the chief complaint of a patient, intakes, outputs, physical information, medications, or demographic information and/or other information relevant to a patient's health. The data may be input from a user or information available from another source, such as a database. The first user may generally be a first point of contact for the patient in the healthcare workflow, for example a nurse, emergency medical technician riding in an ambulance, or other healthcare professional that may gather data about a patient.

Once data is input into the system using the user computers 120, 140, the data may be communicated to the central computer 110. In an embodiment, the central computer 110 may utilize computer software to analyze the data and return available procedure types based on the data. The analysis performed by the computer software may include mapping the data among potential procedures to determine which procedures should be performed based on the data. Procedures that are successfully mapped with the data may be recommended to the user. Alternatively, the computer software may execute on the user computers 120, 140 or some combination of the user computers 120, 140 and the central computer 110. Regardless of location of execution, the computer software analyzes the data and returns available procedure types based on the data. The available procedure types may be displayed in the window 238 in the order entry compartment 230. In an embodiment, the available procedure types may be highlighted for easy identification to a user.

A second user, such as a physician, may review the available procedure types window 238 for the returns of the computer software. The returns of the computer software may be the recommended examinations based on the data available for a patient, for example, data available in window 220. The second user, such as a physician, may add the recommended examinations to the requested procedure window 236. The second user may also add other examinations, such as examinations not recommended by the computer software to the requested procedure window. Once the second user is satisfied with the examinations to be performed, the second user may select the order button 246. Accordingly, a communication is sent to the central computer 110 and communicated to the appropriate units for ordering of the requested examinations.

In such a manner, the decision and diagnosis of a physician is supported by the computer system 100, including computer software, and user interface 200. The computer software may minimize the need for a physician to memorize the available exams for a set of symptoms or minimize the need for a physician to consult a medical reference during an examination. The computer software may also recommend examinations of which the physician may not have thought or recommend new examinations of which the physician is not aware.

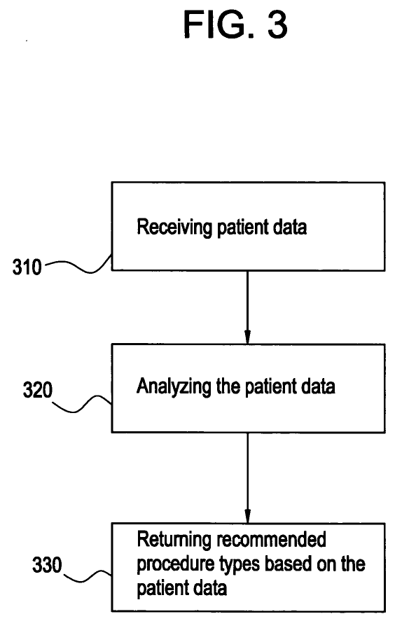

FIG. 3 illustrates a method for supporting a patient workflow computer software. In step 310, the computer software receives patient data. The data may include, for example, medical data, patient symptoms, patient vitals, or patient history. The data may also include, for example, one or more of the chief complaint of a patient, intakes, outputs, physical information, medications, or demographic information. The computer software may receive the patient data as input from a user or from another source, such as a database.

In step 320, patient data is analyzed. The analysis may include mapping the patient data among potential procedures to determine which procedures should be performed based on the patient data. Procedures that are successfully mapped with the patient data may be recommended to the user.

In step 330, the computer software returns the recommended procedure types based on the patient data. The recommended procedure types may be identified and displayed in window 238. A user may add the recommended procedure types to the requested procedures window 236 and order the requested procedures with button 246.

The system and method described above may be carried out as part of a computer-readable storage medium including a set of instructions for a computer. The set of instructions may include a receipt routine for receiving patient data. The data may include, for example, medical data, patient symptoms, patient vitals, or patient history. The data may also include, for example, one or more of the chief complaint of a patient, intakes, outputs, physical information, medications, or demographic information. The computer software may receive the patient data as input from a user or from another source, such as a database.

The set of instructions may include an analysis routine for analyzing the patient data. The analysis may include mapping the patient data among potential procedures to determine which procedures should be performed based on the patient data. Procedures that are successfully mapped with the patient data may be recommended to the user.

The set of instructions may also include a return routine for returning the recommended procedure types based on the patient data. The recommended procedure types may be identified and displayed in window 238. A user may add the recommended procedure types to the requested procedures window 236 and order the requested procedures with button 246.

While the invention has been described with reference to certain embodiments, it will be understood by those skilled in the art that various changes may be made and equivalents may be substituted without departing from the scope of the invention. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims

1. A system for supporting a patient workflow, said system comprising:
a computer system for executing computer software, said computer software analyzing data and returning available procedure types based on said data.

2. The system of claim 1, wherein said data includes medical data.

3. The system of claim 1, wherein said data includes patient symptoms.

4. The system of claim 1, wherein said data includes patient vitals.

5. The system of claim 1, wherein said data includes patient history.

6. The system of claim 1, wherein said data includes one or more of the chief complaint of a patient, intakes, outputs, physical information, medications, or demographic information.

7. The system of claim 1, wherein said computer software includes a user interface, said user interface having a user compartment, a patient information compartment, and an order entry compartment.

8. The system of claim 7, wherein said order entry compartment identifies said available procedure types.

9. The system of claim 8, wherein said order entry compartment provides for ordering said available procedure types.

10. A method for supporting a patient workflow using computer software, said method comprising:
receiving patient data;
analyzing said patient data; and
returning recommended procedure types based on said patient data.

11. The method of claim 10, wherein said patient data includes patient symptoms.

12. The method of claim 10, wherein said patient data includes patient vitals.

13. The method of claim 10, wherein said patient data includes patient history.

14. The method of claim 10, wherein said patient data includes one or more of the chief complaint of a patient, intakes, outputs, physical information, medications, or demographic information.

15. The method of claim 10, wherein said computer software includes a user interface, said user interface having a user compartment, a patient information compartment, and an order entry compartment.

16. The method of claim 15, wherein said order entry compartment identifies said recommended procedure types.

17. The method of claim 15, wherein said order entry compartment provides for ordering said recommended procedure types.

18. A computer-readable storage medium including a set of instructions for a computer, the set of instructions comprising:
a receipt routine for receiving patient data;
an analysis routine for analyzing said patient data; and
a return routine for returning recommended procedure types based on said patient data.

19. The method of claim 18, wherein said patient data includes one or more of the chief complaint of a patient, intakes, outputs, physical information, medications, demographic information, patient vitals, patient symptoms, or patient history.

20. The set of instructions of claim 18, wherein said set of instructions further includes a user interface routine for operating a user interface, said user interface having an order entry compartment.
